(19) United States
(12) Patent Application Publication
(10) Pub. No.: US 2015/0133841 A1
(43) Pub. Date: May 14, 2015

(54) POSTURE LOOP

(71) Applicant: Paul CHEN, Vancouver (CA)

(72) Inventor: Paul CHEN, Vancouver (CA)

(73) Assignee: Paul CHEN, Vancouver (CA)

(21) Appl. No.: 14/079,775

(22) Filed: Nov. 14, 2013

(51) Int. Cl. A61F 5/02

(52) U.S. Cl. CPC A61F 5/026
USPC 602/19

(57) ABSTRACT

A posture loop includes an elastic band or cable having two end portions, a retainer bridge having a bar, the elastic cable includes an intermediate portion engaged with one end portion of the bar, and the end portions of the elastic cable are engaged with the other end portion of the bar, and two buckle members are attached to the end portions of the elastic cable for adjustably coupling the end portions of the elastic cable to the other end portion of the bar and for forming an eight (8)-shaped structure for the posture loop and for engaging onto shoulder portions of a user, The buckle members each include two grooves formed by a rib for engaging with the end portions of the elastic cable respectively.

### BACKGROUND OF THE INVENTION

1. Field of the Invention

The present invention relates to an elastic exercise device or back posture training or exercising or straightening device or posture loop, and more particularly to a back posture training or exercising or straightening device or posture loop including an elastic rope or wire or band or cable supported with a retainer bridge to form an eight (8)-shaped structure or configuration for engaging with or onto the back or shoulder portion of the user and for training or exercising or straightening the shoulder portion and/or the back portion of the user.

2. Description of the Prior Art

Various kinds of typical elastic exercise devices have been developed and provided for conducting or operating various exercise operations and comprise an elastic rope or wire or cable for being operated to exercise various portions or various muscle groups of the user.

For example, U.S. Pat. No. 4,090,706 to Reda, U.S. Pat. No. 4,609,191 to Remme, U.S. Pat. No. 4,733,862 to Miller, U.S. Pat. No. 4,756,527 to Ledbetter, U.S. Pat. No. 4,909,505 to Tee, U.S. Pat. No. 5,318,494 to Santighian, U.S. Pat. No. 5,746,687 to Vial et al., U.S. Pat. No. 5,885,196 to Gvoich, U.S. Pat. No. 6,203,476 to Wang et al., U.S. Patent No. 6,648,804 to Chen, and U.S. Pat. No. 6,866,617 to Chen disclose several of the typical elastic exercise devices each also comprising an elastic rope or wire or cable for being operated to exercise various portions or various muscle groups of the user.

However, the typical elastic exercise devices may only be pulled or operated or actuated to train or exercise the upper or lower muscle groups, but may not be used to train or exercise or straighten the shoulder portion and/or the back portion of the user.

U.S. Pat. No. 4,852,874 to Sleichter, III et al. disclose another typical elastic exercise device also comprising an elastic rope or wire or cable supported with a retainer ring or sleeve for forming an endless loop or for forming an eight (8)-shaped structure or configuration and for being operated to exercise various portions or various muscle groups of the user, and for training or exercising the upper or lower muscle groups of the user.

However, the typical elastic exercise devices may only be used or pulled or operated or actuated to train or exercise the upper or lower muscle groups of the user, but may not be used to train or exercise or straighten the shoulder portion and/or the back portion of the user.

The present invention has arisen to mitigate and/or obviate the afore-described disadvantages of the conventional elastic exercise devices.

### SUMMARY OF THE INVENTION

The primary objective of the present invention is to provide a posture loop including an elastic rope or wire or band or cable supported with a retainer bridge for forming an endless loop or for forming an eight (8)-shaped structure or configuration and for training or exercising or straightening the shoulder portion and/or the back portion of the user.

The other objective of the present invention is to provide a posture loop including an elastic rope or wire or band or cable having two ends adjustably connected to the retainer bridge for applying an adjustable or changeable resilience or resilient force onto the user, and for allowing the posture loop to be suitably and selectively engaged onto or with the shoulder portions of the users of different sizes or dimensions or standards.

In accordance with one aspect of the invention, there is provided a posture loop comprising a retainer bridge including a bar having a first end portion and a second end portion, an elastic cable including an intermediate portion engaged with the first end portion of the bar, and including a first end portion and a second end portion engaged with the second end portion of the bar respectively, and two buckle members attached to the first and the second end portions of the elastic cable respectively for slidably and adjustably coupling the first and the second end portions of the elastic cable to the second end portion of the bar and for forming an eight (8)-shaped structure for the posture loop and for engaging onto shoulder portions of a user.

The bar of the retainer bridge includes a pathway formed in the first end portion of the bar for slidably receiving and engaging with the intermediate portion of the elastic cable and for slidably and adjustably attaching or coupling the first end portion of the bar to the intermediate portion of the elastic cable.

The bar of the retainer bridge includes two channels formed in the second end portion of the bar for slidably receiving and engaging with the first and the second end portions of the elastic cable respectively and for adjustably coupling the first and the second end portions of the elastic cable to the second end portion of the bar.

The buckle members each include a rib formed or provided in the middle or intermediate portion thereof for forming two grooves therein and for slidably receiving and engaging with the first and the second end portions of the elastic cable respectively, and thus for slidably attaching or coupling the first and the second end portions of the elastic cable to the buckle members.

The bar includes two channels formed in the second end portion of the bar, the first and the second end portions of the elastic cable are engaged through the grooves and over the rib of the buckle members respectively, and then engaged through the channels of the bar respectively, and then coupled to the ribs of the buckle members respectively for slidably and adjustably coupling the first and the second end portions of the elastic cable to the second end portion of the bar.

Two cushioning members may further be provided and each include a passage formed therein for slidably receiving and engaging with the intermediate portion of the elastic cable, and the cushioning members each include a pad for softly engaging with the user.

The elastic cable is separated into a first segment and a second segment with the cushioning members, and the bar of the retainer bridge is engaged between the first and the second segments of the elastic cable for separating the first and the second segments of the elastic cable from each other.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a rear plan schematic view illustrating the operation of a posture loop in accordance with the present invention;

FIG. 2 is a partial perspective view illustrating the operation of the posture loop;

FIG. 3 is a partial exploded view of the posture loop;

FIG. 4 is a plan schematic view of the posture loop;

FIG. 5 is an enlarged partial perspective view of the posture loop; and

FIG. 6 is a partial top plan schematic view illustrating the operation of the posture loop.

### DETAILED DESCRIPTION OF THE PREFERRED EMBODIMENT

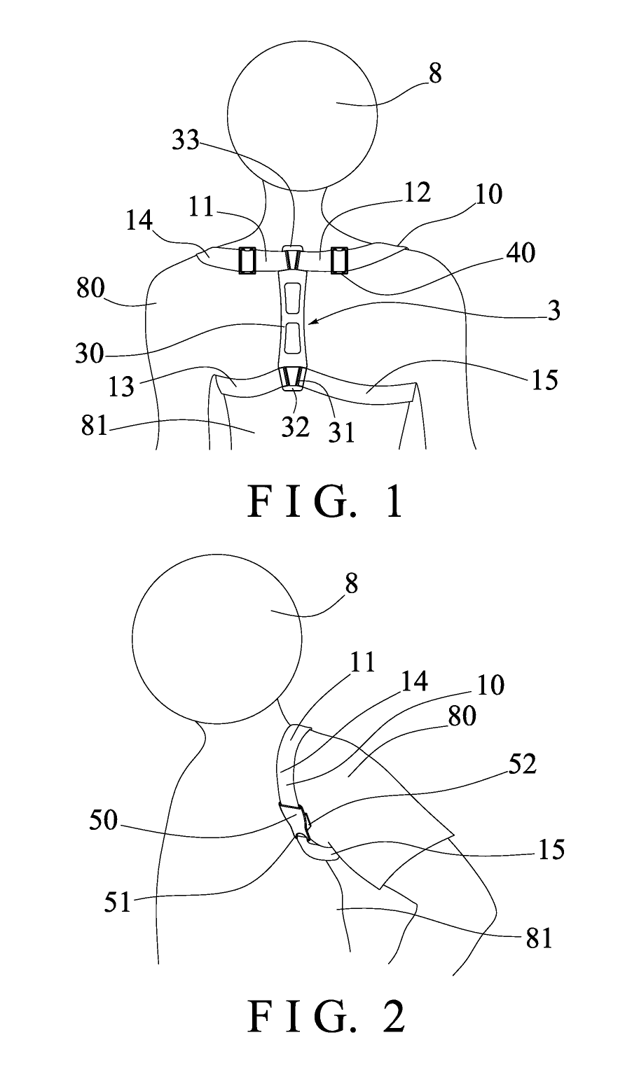

Referring to the drawings, and initially to FIGS. 1-4, a posture loop in accordance with the present invention comprises an elastic rope or wire or band or cable 10 having two end portions 11, 12 to be coupled or connected together for forming an endless loop, and having a middle or intermediate portion 13 coupled or connected together with a retainer bridge 3 for forming an eight (8)-shaped structure or configuration and for engaging with or onto the shoulder portions 80 of a user 8 (FIGS. 1-2), and for training or exercising or straightening the shoulder portion 80 and/or the back portion 81 of the user 8, or for training or correcting the posture of the user 8.

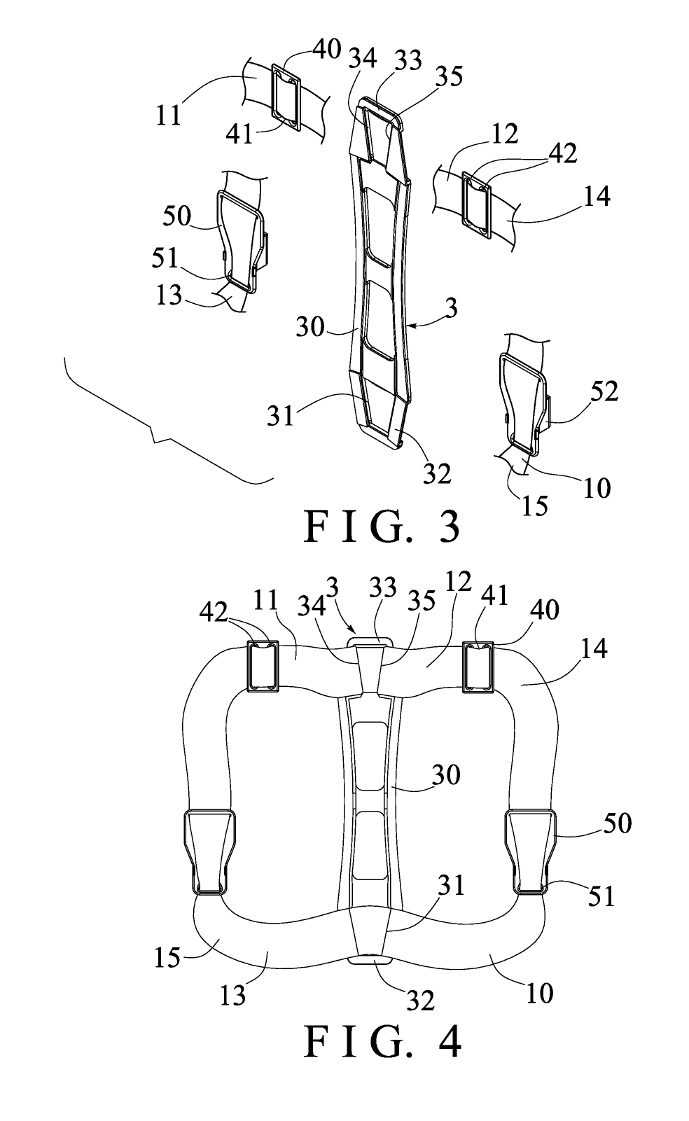

For example, as shown in FIGS. 3-6, the retainer bridge 3 is threaded or attached or engaged with or onto the elastic cable 10 for forming an eight (8)-shaped structure or configuration for the posture loop and thus for allowing the posture loop to be attached or mounted or secured or engaged with or onto the shoulder portion 80 and/or the back portion 81 of the user 8. The retainer bridge 3 includes a longitudinal bridge body or rod or beam or bar 30 having a lower groove or channel or slot or passage or pathway 31 formed therein, such as formed in the lower portion or first end portion 32 thereof for slidably receiving or threading or engaging with the middle or intermediate portion 13 of the elastic cable 10 and thus for attaching or mounting or securing or coupling the lower portion or first end portion 32 of the bar 30 of the retainer bridge 3 to the elastic cable 10.

The bar 30 of the retainer bridge 3 includes an upper portion or second end portion 33 having one or more (such as two) passages or slots or pathways or grooves or channels 34, 35 formed therein for slidably receiving or threading or engaging with the two end portions 11, 12 of the elastic cable 10 and for adjustably coupling the end portions 11, 12 of the elastic cable 10 to the upper portion or second end portion 33 of the bar 30 of the retainer bridge 3. Two latch rings or latch elements or buckle members 40 are provided for adjustably attaching or mounting or securing or coupling the end portions 11, 12 of the elastic cable 10 to the upper portion or second end portion 33 of the bar 30 of the retainer bridge 3, and each of the buckle members 40 include a beam or bar or rib 41 disposed or provided or engaged therein for forming or defining one or more (such as two) passages or slots or channels or pathways or grooves 42 therein and for threading or engaging with the end portions 11, 12 of the elastic cable 10.

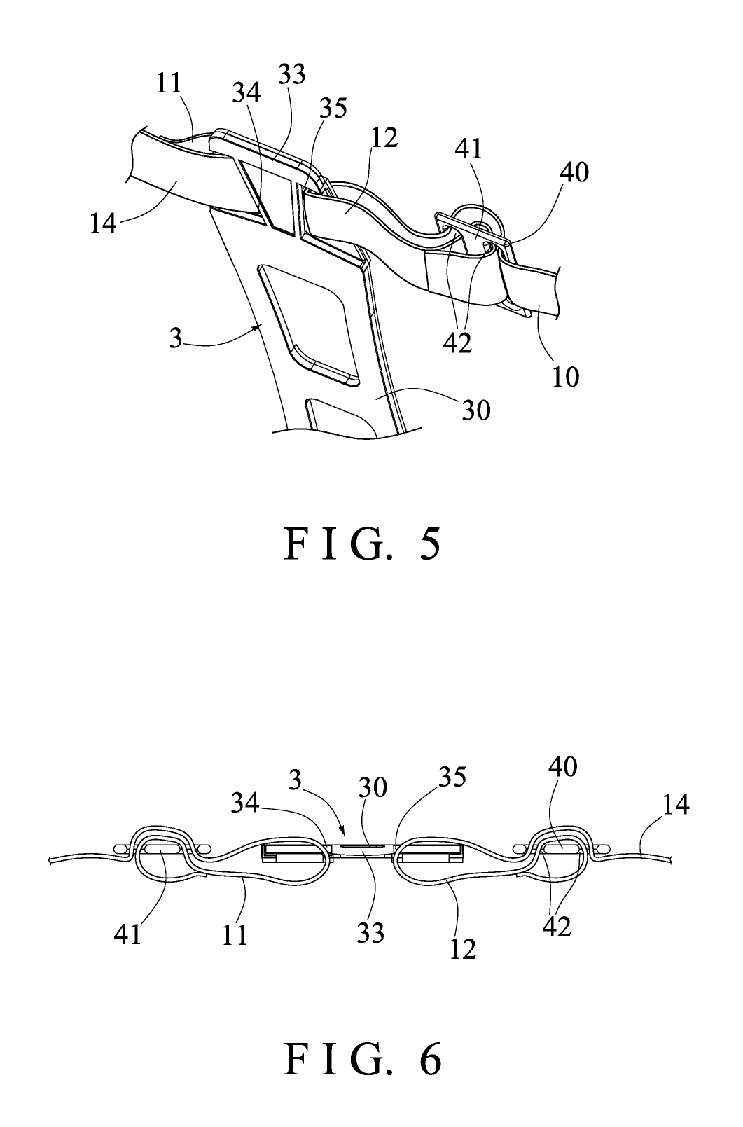

As shown in FIGS. 5 and 6, the end portions 11, 12 of the elastic cable 10 are engageable through the grooves 42 and over the rib 41 of the buckle member 40 respectively, and then engaged through the channel 34, 35 of the bar 30 of the retainer bridge 3, and then attached or mounted or secured or coupled to the rib 41 of the buckle member 40 for adjustably attaching or mounting or securing or coupling the end portions 11, 12 of the elastic cable 10 to the second end portion 33 of the bar 30 and thus for adjusting the length of the endless loop of the elastic cable 10 or the eight (8)-shaped structure or configuration of the posture loop and thus for allowing the posture loop to be suitably and selectively engaged onto or engaged with the shoulder portions 80 and/or the back portion 81 of the users 8 of different sizes or dimensions or standards.

Two pads or cushioning members 50 are attached or mounted or secured onto the elastic cable 10, such as the middle or intermediate portion 13 of the elastic cable 10, and each include a pathway or channel or groove or slot or passage 51 formed therein for slidably receiving or threading or engaging with the middle or intermediate portion 13 of the elastic cable 10 and for slidably attaching or mounting or securing or retaining the cushioning members 50 to the middle or intermediate portion 13 of the elastic cable 10. The cushioning members 50 each include a soft pad 52 attached or mounted or secured thereto and made of soft or spongy or resilient materials for softly and comfortably engaging with or onto the shoulder portion 80 and/or the back portion 81 of the user 8.

The elastic cable 10 may be spaced or separated by the two cushioning members 50 into two portions or segments 14, 15, such as an upper or first portion or segment 14 having the two end portions 11, 12 of the elastic cable 10, and a lower or second portion or segment 15 having most of the middle or intermediate portion 13 of the elastic cable 10. The retainer bridge 3 may thus be used for extending or spacing or retaining or supporting or disengaging or separating the segments 14, 15 of the elastic cable 10 from each other for thus forming the eight (8)-shaped structure or configuration for the posture loop, and may include a lower base element and an adjustable or movable element (not illustrated) attached or mounted or secured together or engaged with each other for forming or defining an adjustable structure or configuration for the retainer bridge 3 and for allowing the posture loop to be suitably and selectively engaged onto or with the shoulder portions 80 and/or the back portion 81 of the users 8 of different sizes or dimensions or standards.

In operation, as shown in FIGS. 1-2, the posture loop includes an eight (8)-shaped structure or configuration for selectively engaging with or onto the shoulder portions 80 and/or the back portion 81 of the user 8, and for training or exercising or straightening the shoulder portion 80 and/or the back portion 81 of the user 8. As shown in FIGS. 5-6, the two end portions 11, 12 of the elastic cable 10 are movable or adjustable relative to the upper portion or second end portion 33 of the bar 30 of the retainer bridge 3 for adjusting the length of the elastic cable 10, and in order to adjust the resilience or spring biasing force of the elastic cable 10 to the user 8, and/or for allowing the posture loop to be suitably and selectively engaged onto or with the shoulder portions 80 and/or the back portion 81 of the users 8 of different sizes or dimensions or standards. The retainer bridge 3 itself may also include an adjustable structure or configuration (not illustrated) for adjusting the distance between the segments 14, 15 of the elastic cable 10 and thus for allowing the posture loop to be suitably and selectively engaged onto or with the shoulder portions 80 and/or the back portion 81 of the users 8 of different sizes or dimensions or standards.

Accordingly, the posture loop in accordance with the present invention includes an elastic rope or wire or cable having two end portions coupled together for forming an endless loop and having a middle or intermediate portion supported with a retainer bridge for forming an eight (8)-shaped structure or configuration and for training or exercising or straightening the back portion of the user, the ends of the elastic band or cable are adjustably connected together for applying an adjustable or changeable resilience or resilient force onto the user.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A posture loop comprising:
a retainer bridge including a bar having a first end portion and a second end portion,
an elastic cable including an intermediate portion engaged with said first end portion of said bar, and including a first end portion and a second end portion engaged with said second end portion of said bar respectively, and
two buckle members attached to said first and said second end portions of said elastic cable respectively for adjustably coupling said first and said second end portions of said elastic cable to said second end portion of said bar and for forming an eight (8)-shaped structure for said posture loop and for engaging onto shoulder portions of a user.

2. The posture loop as claimed in claim 1, wherein said bar of said retainer bridge includes a pathway formed in said first end portion of said bar for slidably receiving and engaging with said intermediate portion of said elastic cable.

3. The posture loop as claimed in claim 1, wherein said bar of said retainer bridge includes two channels formed in said second end portion of said bar for slidably receiving and engaging with said first and said second end portions of said elastic cable respectively and for adjustably coupling said first and said second end portions of said elastic cable to said second end portion of said bar.

4. The posture loop as claimed in claim 1, wherein said buckle members each include a rib provided therein for forming two grooves therein and for engaging with said first and said second end portions of said elastic cable respectively.

5. The posture loop as claimed in claim 4, wherein said bar includes two channels formed in said second end portion of said bar, said first and said second end portions of said elastic cable are engaged through said grooves and over said rib of said buckle members respectively, and then engaged through said channels of said bar respectively, and then coupled to said ribs of said buckle members respectively for adjustably coupling said first and said second end portions of said elastic cable to said second end portion of said bar.

6. The posture loop as claimed in claim 1 further comprising two cushioning members each including a passage formed therein for slidably receiving and engaging with said intermediate portion of said elastic cable, and said cushioning members each including a pad for engaging with the user.

7. The posture loop as claimed in claim 6, wherein said elastic cable is separated into a first segment and a second segment with said cushioning members, and said bar of said retainer bridge is engaged between said first and said second segments of said elastic cable for separating said first and said second segments of said elastic cable from each other.
